(19) United States
(12) Patent Application Publication
(10) Pub. No.: US 2010/0240147 A1
(43) Pub. Date: Sep. 23, 2010

(54) HIGH SENSITIVITY NANOTECHNOLOGY-BASED MULTIPLEXED BIOASSAY METHOD AND DEVICE

(76) Inventors: Gianfranco Liguri, Firenze (IT); Francesco Trisolini, Montevarchi (IT)

Correspondence Address: MCGLEW & TUTTLE, PC, P.O. BOX 9227, SCARBOROUGH STATION, SCARBOROUGH, NY, 10510-9227, US

(21) Appl. No.: 12/680,310

(22) PCT Filed: Sep. 26, 2008
(86) PCT No.: PCT/IB08/02526

§ 371 Date: Mar. 26, 2010

(30) Foreign Application Priority Data
Sep. 26, 2007 (IT) PO2007A000023

(51) Int. Cl. G01N 33/566; G01N 30/00

(52) U.S. Cl. 436/501; 422/69

(57) ABSTRACT

In a method and device for high sensitivity analysis of multiple bioassays plurality of nanoparticles (N) is dispersed in a fluid sample, at least one probe (C) of a first type having affinity for at least one analyte being attached to each nanoparticle; a plurality of probes of at least a second type having affinity for the analyte is attached to the internal surface (S) of the container; means are provided for detecting the signal generated by the adhesion of the nanoparticles to the internal surface of the reactor, brought about by the interaction of the analyte with the nanoparticle-bound probes and that of the analyte with the probes attached to the internal surface. The method and the device are particularly effective for detecting different genotypes of human papilloma virus (HPV).

### TECHNICAL FIELD

This invention refers to a method and a device for the high sensitivity analysis of multiple analytes in biological samples. To go into detail, the invention refers to a method and a device for the detection of complementary pairs of molecules, such as nucleic acid-nucleic acid (e.g. DNA-DNA, DNA-RNA, RNA-RNA) or protein-protein (e.g. antigen-antibody) pairs.

### PRIOR ART

It is well-known that during the last twenty years, molecular biology techniques have generated growing interest in the fields of biology and medicine. In particular, there is a strong interest in developing new bioassay techniques for a wide variety of applications, such as gene identification, gene mapping and DNA sequencing in medicine and in other diagnostic fields.

Amongst the more well-known techniques, Polymerase Chain Reaction (PCR) and its related modifications has been widely used both in research and in clinical diagnostics to specifically amplify and analyze traces of nucleic acids whose initial and terminal nucleotide sequences are known. This technique relies on the ability of the DNA polymerase enzyme to synthesize a new DNA strand from denatured DNA at certain temperatures. Ligase Chain Reaction (LCR), which relies on the action of a thermostable ligase, represents the most common variation of PCR. These techniques are very sensitive and reliable, but are also very expensive and time-consuming. Moreover, they require skilled technicians and are not very easy to automate. Quantitative PCR (Q-PCR), which is based on the amplification of target DNA, is another common modification of PCR, but is only able to generate semi-quantitative data (number of copies of a DNA sequence in a sample) and can be affected by the presence of inhibitors and low copy number templates.

ELISA (Enzyme-Linked Immunosorbent Assay) and its related modifications is another technique that is frequently used in diagnostics. It is a type of immunologic analysis that, in biochemistry, is used to detect the presence of an antigen that is characteristic of a particular pathogenic organism, or to measure the concentration of antibodies in blood plasma (as in AIDS tests, for example). This technique relies on specific antigen-antibody reactions that are made visible by various procedures. In the last few years, many other ELISA-related techniques have been developed. One of them is the “Two-hybrid capture” technique, developed by Digene Corp. for the detection of human papilloma virus (HPV), which is able to detect RNA:DNA hybrids using an amplified chemiluminescent signal. These ELISA-related techniques, though sensitive and reliable, do not permit ‘multiplexing’ (the simultaneous detection of different subtypes (e.g. HPV subtypes) in the same reactor). The accuracy of such techniques can also be affected by the fluorescent signal used in detection, which tends to be fairly insensitive, by the low stability of dyes and by the influence of the physicochemical environment on signal intensity. Detection is often expensive, too.

At the start of the 1990s, DNA microarrays were becoming more important for parallel detection of DNA molecules, particularly in the field of biomedical research. A DNA microarray is a collection of microscopic DNA probes arrayed on a solid surface, such as glass or plastic, that bind to chemically suitable, complementary targets that have been previously amplified and tagged with a fluorescent molecule, which permits detection of fluorescent signals generated upon hybridization. These techniques therefore permit multiplexing, but require prior target DNA amplification to ensure optimal sensitivity, as in the case of RT-PCR-amplified mRNA detection. As such, they are affected by the aforementioned drawbacks of PCR. Microarrays also require very expensive scanners and sophisticated detection systems, which consist of specific focalized lasers that reveal every single micro-spot. Moreover, they are not easily automated and are therefore not very useful in diagnostics. In addition to this, they are also affected by the aforementioned limitations of fluorescent detection.

In recent years, new detection techniques derived from the well-known “Latex Agglutination” assay have been developed, with the aim of overcoming the analytical drawbacks associated with fluorescence-based techniques and PCR. In this case, the surface of the latex particles is coated with antibody (or antigen). When a suspension containing the complementary antigen (or antibody) to be detected is added to the particle suspension, it causes visible agglutination that allows the specific antigen or antibody to be detected by a dimensional scale “shift”. However, these methods do not permit multiplexing, they require larger particles (micrometres in size) and are often only applied to specific antigens or antibodies. Numerous patents from this field can be found (U.S. Pat. No. 6,200,820, U.S. Pat. No. 5,589,401, U.S. Pat. No. 7,122,384, U.S. Pat. No. 7,169,556, U.S. Pat. No. 5,175,112), but most of these refer to very complex and mainly non-quantitative techniques.

### DISCLOSURE OF THE INVENTION

Thus, the aim of this invention is to deliver an analytical biotechnology technique that is able to carry out detection of complementary pairs of molecules, such as nucleic acid-nucleic acid (DNA-DNA, DNA-RNA, RNA-RNA) or protein-protein pairs, without the drawbacks of other techniques.

In particular, the object of the invention is to provide a method and a device for the simultaneous and quantitative detection of different subtypes (multiplexing) of a given biological target system (virus, genetic mutations, etc.).

The aforementioned aims are achieved using a method and a device that exploits the interaction of non-biological nanoparticles with biological structures (such as nucleic acids and proteins) to determine dimensional increases, thereby permitting the detection of a biological target (virus, genetic mutations, etc.).

In particular, the proposed method and device make use of a microarray architecture of nanoparticles linked to molecular probes or specific antibodies, which are adsorbed onto a solid surface and preferably arranged in a monolayer. The aim of the system is to specifically detect complementary pairs of molecules, such as protein-protein pairs (e.g. antigen-antibody complexes) and DNA-DNA or DNA-RNA pairs (e.g. probe-target complexes). In addition to the use of fluorescent signals to detect and quantify these pairs, focalized laser sources coupled to a system of photosensors can preferably be used. Various detection strategies can then be exploited, such as image analysis or light scattering.

The advantages and technical characteristics of the invention will become more apparent from the following detailed description of a non-limiting example embodiment of it.

### BRIEF DESCRIPTION OF THE DRAWINGS

In the drawings:

FIG. 1 schematically illustrates the methodological purpose of the invention;

FIG. 2 schematically illustrates the application of the invention to the detection of several subtypes of a biological system;

FIG. 3 shows the magnification of detail A from FIG. 2.

### PREFERRED EMBODIMENT OF THE INVENTION

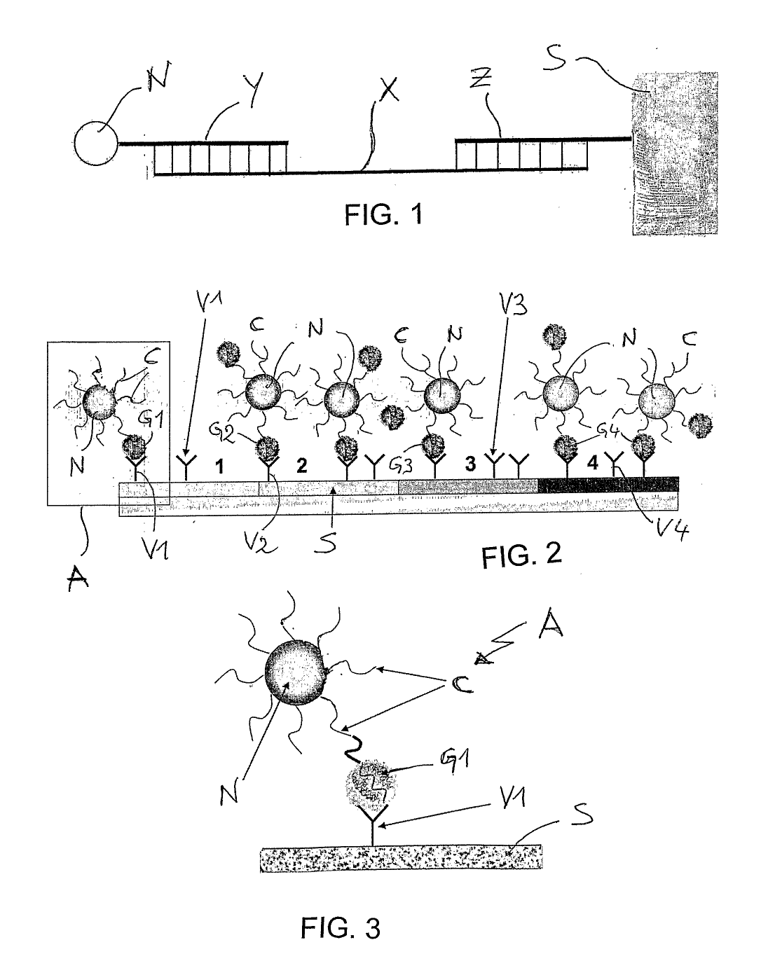

With reference to FIG. 1, to enable the detection of element X of a complementary pair of molecules (the target), binding to two other components Y,Z of the complementary element of the other pair is determined (in the illustrated example, two elements complementary to two different terminal portions of the target), one attached to a nanoparticle N and the other to a solid support S.

More specifically, in conformity with the invention, latex nanoparticles N, that are monodispersed in the suspension under test, specifically bind to the target X through complementary molecular probes Y that are attached to the nanoparticles. These nanoparticle-probe-target complexes diffuse by Brownian motion from the liquid suspension to the solid-liquid interface and dock onto the solid surface S (e.g. one of the internal surfaces of the tube containing the suspension) as a monolayer, resulting in the target X binding to other specific molecular probes Z (which are complementary to other portions of the target), that are also adsorbed onto the solid surface.

In this way, by docking different probes onto different areas of the solid surface (as shown below), an array of particles is created, with each area characterised by a particular probe-particle complex, thereby enabling the specific characterisation of several subtypes of a given biological system (multiplexing).

The proposed method guarantees rigidity and reliability, as well as rapid analysis and automation. Moreover, less time and money will be required to train personnel, compared to previous techniques.

Very sensitive analysis is obtained, not by a quantitative increase in material, as with PCR, but by exploiting the amplification signal that is generated when shifting from a molecular scale (nucleic acid or protein) to a microscopic one (latex nanoparticles): in fact, the “shift” in the dimensional scale is two- to three-fold in magnitude. An additional process that increases the sensitivity of the test is the concentration effect that occurs when monodispersed particles diffuse though the liquid suspension and dock onto the solid surface as a monolayer.

Consequently, the method and the device according to the present invention enable two crucial aims in the medical diagnostic field to be fulfilled. The first of these is signal enhancement, in addition to better use of the solid space and selective concentration of the molecule to be analysed in a two-dimensional state, and secondly, the ability to carry out more simultaneous tests, in the same reactor and on the same sample (multiplexing), resulting in a considerable saving of time, money and biological material.

In addition to those mentioned previously, this last feature, in particular, is especially advantageous when it comes to detecting genetic mutations in human samples and can also be used to genotype viruses that are dangerous to humans.

In FIGS. 2 and 3, the detection of different genotypes (four in this case) of Human Papilloma Virus (HPV) is schematically illustrated using a method and a device that conforms to the proposed invention.

In this example, the genome (G1-G4) of the various HPV subtypes is the target to be detected. The probes (C) attached to the nanoparticles (N) are DNA probes that are complementary to the conserved regions of the HPV genome (and are therefore able to detect any HPV subtype), whereas the probes (V1-V4) attached to the solid support (S) are DNA probes that are complementary to the variable regions of the HPV genome (therefore, they can specifically detect a particular HPV subtype). As a consequence, this permits the spatial detection of various subtypes (multiplexing).

The system foresees the use of a plastic container (e.g. a tube), on one of whose internal surfaces (S) an area of approximately 1 cm² is selected and sub-divided into different cells (1-4). A different oligonucleotide (V1-V4), corresponding to a particular DNA probe that is complementary to the variable part of the genome of a specific HPV genotype, is attached to each cell.

A suspension containing latex nanoparticles (N) that are roughly 100-300 nm in size, which are linked to DNA probes (C) (previously attached to the nanoparticles) that are specific for the conserved portion of the HPV genome and, as a consequence, related to the various HPV subtypes, will then be added to the tube.

The biological sample is added to the tube so that qualitative and quantitative measurements of HPV can be made. In the presence of a specific target (e.g. G1) (i.e. the presence of a molecule of a certain HPV subtype), binding occurs between the target and a nanoparticle (N), through its conserved DNA portion (C), and with portion (1) of the internal surface (S) of the tube, through its variable DNA portion (V1), resulting in docking of the nanoparticles onto the internal surface of the tube (FIG. 3).

After washing (if necessary) to eliminate any unbound nanoparticles, qualitative detection of the various HPV subtypes present in the sample will be carried out (e.g. using light scattering techniques, image analysis or evanescent wave techniques) to determine the spatial distribution of nanoparticles that are bound to surface-attached type-specific oligonucleotide probes.

By producing calibration curves derived from experimental data, as well as by using a specific mathematical algorithm, it is also possible to perform quantitative detection of each HPV subtype present in the sample that was tested. The probabilistic and statistical correlation of the effective concentration of each specific subtype within the sample with the number of specific nanoparticles that are bound in a monolayer to the solid support avoids the possibility of under- or overestimating one or more subtypes.

This invention is subject to numerous modifications and variations, all of which fall within the limits of the original concept. Furthermore, all the details can be formed from technically equivalent elements.

1. A high sensitivity multiple bioassay method, comprising the following steps:
providing a plurality of nanoparticles;
applying at least one probe of a first type, that has affinity for at least one analyte, to each nanoparticle;
applying a plurality of probes of at least one second type, that have affinity for said at least one analyte, to the surface of a support;
mixing said plurality of nanoparticles with a fluid sample thereby determining the linkage of one or more of said nanoparticles to said analyte, when contained in the sample, as a result of the binding occurring between the probe of the first type and the analyte;
bringing said analyte-linked nanoparticles into contact with said support to bring about attachment to the surface as a result of the binding occurring between the probes of said second type and said analyte;
detecting said analyte by analyzing the signal generated by said surface-bound nanoparticles.

2. A method according to claim 1, wherein said probes of the first type are complementary to a conserved region of said analyte.

3. A method according to claim 1, wherein said nanoparticles consist of spherical particles that are 1-1000 nm of size, are made of organic polymers or co-polymers or are siliceous or metallic in nature (e.g. silica, golden or silver particles).

4. A method according to claim 1, wherein said probes of the second type are complementary to variable regions of said analyte.

5. A method according to claim 1, wherein the surface of said support is divided into a plurality of areas and at least one probe, that is complementary to a different variable region of said analyte, is applied to each area, thereby resulting in the docking of nanoparticles linked to different subtypes of the analyte in different areas.

6. A high sensitivity multiple bioassay device, comprising
a receptacle in which a fluid sample can be analyzed;
a plurality of nanoparticles dispersed in the fluid, with each nanoparticle attached to at least one probe of a first type having affinity for at least one analyte;
a plurality of probes of at least a second type having affinity for said analyte and being attached to the surface of at least one wall of the receptacle;
means for detecting the signal generated by the nanoparticles that dock to the surface of the wall, resulting from the bindings occurring between the nanoparticle-bound probes and the analyte and between the analyte and the probes attached to the surface.

7. A device according to claim 6, wherein the surface of said wall is divided into a plurality of areas, with each area being linked to at least one probe that is complementary to a different variable region of said analyte, thereby resulting in the docking of nanoparticles linked to different subtypes of the analyte in different areas.

8. A method according to claim 1, wherein different genotypes of Human Papilloma Virus (HPV) are detected.

9. A device according to claim 6, wherein different genotypes of Human Papilloma Virus (HPV) are detected.

10. A method according to claim 2, wherein said nanoparticles consist of spherical particles that are 1-1000 nm of size, are made of organic polymers or co-polymers or are siliceous or metallic in nature (e.g. silica, golden or silver particles).
